(19) United States
(12) Patent Application Publication
(10) Pub. No.: US 2009/0312646 A1
(43) Pub. Date: Dec. 17, 2009

(54) OPTICAL DETECTION OF SEIZURE, A PRE-SEIZURE STATE, AND CEREBRAL EDEMA AND OPTICAL FIBER DETECTION OF THE SAME

(75) Inventors: Devin Binder, Newport Beach, CA (US); Christopher Owen, Long Beach, CA (US); Marlon Mathews, Anaheim, CA (US)

Correspondence Address: Law Offices of Daniel L. Dawes;Dawes Patent Law Group, 5200 Warner Blvd, Ste. 106, Huntington Beach, CA, 92649, US

(73) Assignee: The Regents of the University of California, Oakland, CA (US)

(21) Appl. No.: 12/334,478

(22) Filed: Dec. 14, 2008

Related U.S. Application Data
(63) Continuation-in-part of application No. 12/208,783, filed on Sep. 11, 2008, now abandoned.
(60) Provisional application No. 60/972,136, filed on Sep. 13, 2007.

(51) Int. Cl. A61B 6/00

(52) U.S. Cl. 600/478

(57) ABSTRACT

A method for using optical parameters to monitor for a physiological event and/or a state prior to the physiological event includes the steps of: illuminating neural tissue with diagnostic light of a predetermined frequency at a predetermined location; detecting magnitude of optical scattering by neural tissue of the diagnostic light as a function of time; and determining a signature signal of the optical scattering of the diagnostic light before the physiological event in the neural tissue becomes clinically manifested. An apparatus includes a source of diagnostic light of a predetermined frequency for illuminating neural tissue at a predetermined location, a detector of optical scattering and/or optical absorption by neural tissue of the diagnostic light as a function of time, and a signal processor for determining a signature signal of the optical scattering and/or optical absorption of the diagnostic light before the physiological event becomes clinically manifested.

### RELATED APPLICATIONS

The present application is related to U.S. Provisional Patent Application Ser. No. 60/972,136, filed on Sep. 13, 2007, which is incorporated herein by reference and to which priority is claimed pursuant to 35 USC 119.

### BACKGROUND OF THE INVENTION

1. Field of the Invention

The invention relates to methods of detecting neural seizures and brain tissue edema and apparatus used for the same.

2. Description of the Prior Art

Intrinsic optical imaging (IOS) has been used for decades to map neuronal activity and seizures in cerebral cortex. It is limited insofar as it detects only changes in diffuse reflectance which primarily arise from blood perfusion changes. Further, perfusion change occurs as a result of seizure activity and thus occurs after seizure onset. Our data indicate that scattering changes, by comparison, are associated with cellular changes which begin before a seizure occurs. This is supported by previous work by Binder et. al. in which fluorescence recovery techniques demonstrated a constriction in the extra-cellular space prior to seizure onset.

Seizure activity causes neurons and glial cells to undergo changes which alter the way light propagates through brain tissue. Currently seizures are detected from deep brain structures with the use of electroencephalography (BEG). EEG recordings are made through electrodes placed on the scalp, on the brain surface, or inserted deep in the brain. EEG works through amplification of minute voltage potentials generated in brain tissue. As such it has inherently low signal to noise ratio and requires controlled conditions for optimal signal detection. Optical changes occur before seizures manifest on EEG, making an optical paradigm ideally suited as an early warning detector. Currently, attempts are underway to improve the early detection sensitivity of EEG through complex mathematical manipulations of EEG data, though at the current state of the art it is not possible to reliably predict seizures through real-time analysis of EEG signals.

There is currently no way to detect seizures optically in deep brain structures. Previous optical techniques have been limited to absorption, related changes, and detection at the cortical surface. Detection of deep brain seizure foci is restricted to EEG detection. The disadvantages of these techniques are described above.

Others have reported development of a variety of fiber-optic based probes that detect large scattering differences between gray matter and white matter to allow anatomic localization. Giller, U.S. Pat. No. 4,623,789 describes such a device to be used to aid stereotactic localization in animals to aid research. However no such device has been described for detection of scattering, absorption, or reflectance changes associated with physiologic events.

U.S. Pat. No. 6,526,297 describes a non-invasive device which uses optical scattering measurements to measure changes in neuronal activation for assessing level of anesthesia. However, since the device measures through the skin and samples a large segment of brain, it is not suitable for measuring a seizure focus, typically a small, discrete and deep-seated area of tissue.

Cerebral edema, or brain swelling, can result from various pathological processes, including traumatic injury, stroke, tumor, infection, or surgical manipulation of brain tissue. Cerebral edema is an early and significant contribution to the increase in intracranial pressure associated with these pathologic processes. Swelling of cells and the interstitial space alters the propagation of light through brain tissue.

Probes do exist that use an electrochemical technique for measuring brain tissue oxygenation for clinical management of head injury (U.S. Pat. No. 6,068,743), however the device does not utilize optical signals or provide measurements of cerebral edema. Fiber optic pressure transducers used in intracranial pressure monitors such as manufactured by Integra of Plainsboro, N.J., under the Camino® brand have also been utilized.

There is currently no known device capable of directly measuring cerebral edema. The pathologic sequelae of brain swelling are currently assessed through pressure transducers to measure intracranial pressure, and tissue oxygen saturation probes. However, because of brain compliance and blood flow auto-regulatory mechanisms, both increased intracranial pressure and brain hypoxia are late changes that may occur long after swelling begins.

### BRIEF SUMMARY OF THE INVENTION

We have discovered particular optical parameters which are useful in detection of the seizure and pre-seizure state in the brain. Specifically, we have demonstrated that the degree of optical scattering by neural brain tissue decreases before seizures are detected with electroencephalography (EEG), and before becoming manifested clinically. The optical detection of scattering changes allows us to build an apparatus which can predict and record seizures before they occur and trigger interventions to prevent or abort them. Optical apparatus capable of detecting these scattering changes can also be used to image and map regions of brain tissue undergoing seizure. The configuration of our apparatus allows reliable detection of the pre-seizure state earlier than any previously described system, either optical or electrographic.

The fundamental principle underlying these processes is the small but detectable changes in neural architecture that results from water and ion migration preceding and during brain electrical activity. These changes are most pronounced before and during seizures. Water and ion fluxes likely cause changes in cell volume and the extracellular space which decreases optical scattering through the affected tissue.

The precise arrangement of the source and detector fibers in our apparatus allows for delineation between signal changes due to altered perfusion and those due to changes in cellular architecture, e.g. swelling. However scattering changes can only be detected at small source-to-detector separations. By coupling a source and a detector to fibers, measurements may be obtained from anywhere the fibers are inserted, including deep brain structures.

Seizure detection through optical fibers provides better signal to noise and less susceptibility to interference and movement artifact than EEG. It allows for optical signals to be obtained from anywhere in the brain into which fibers may be inserted, while current optical systems are limited to within a few millimeters of the cortical surface.

Fiber based probes for detection of scattering changes can be implanted into seizure foci of patients with epilepsy and used as early warning devices. Such probes also represent the afferent limb of potential closed loop devices for seizure detection and early termination or prevention.

There is currently no method to predict seizure onset using optical techniques. Prediction of seizure onset provides clinical opportunities for therapeutic interventions for prevention or early arrest of clinical seizures. Seizure detection through measurement of optical scattering provides a reliable method of seizure prediction which has not hitherto been available. This process can be utilized by a variety of optical devices which are capable of measuring scattering coefficients, well known to those with ordinary skill in the art, and is not limited to the illustrated embodiment. The detection of the pre-seizure state has potential uses for seizure early warning systems, closed-loop seizure termination and prevention devices, improved intra-operative mapping of epileptic foci, and optical recording of seizure activity.

We have developed a methodology and apparatus comprising a light source and detector coupled to thin optical fibers selected to minimize invasive damage upon penetration within the tissue. When inserted into the brain, the fibers can be employed to detect alterations in the scattering coefficient of the tissue at various wavelengths, as well as water concentration dependent changes in the absorption coefficient that occur as edema develops.

This methodology or apparatus is also adaptable to a clinically functional implantable probe for direct measurement of cerebral edema in a variety of neurological and neurosurgical conditions. It can provide early warning of pathologic brain swelling well before the currently measurable late sequelae of increased intracranial pressure or hemodynamic changes.

A noninvasive optical detection apparatus utilizing a diode laser at 850 nm used as source and a photomultiplier used as detector has demonstrated efficacy for detecting scattering changes associated with cerebral edema through an intact mouse skull. Such an apparatus, however, is greatly facilitated by the thin skull of the mouse and is not easily translatable to human patients. The thin optical fibers of our apparatus allow the probe to be co-inserted alongside a ventricular drainage catheter in human treatments, which is often placed for diagnostic and therapeutic purposes in human neurosurgical interventions.

Direct detection of cerebral edema allows measurement of one of the primary physiologic events in the cascade of neurologic deterioration from a variety of causes. Earlier detection could allow potentially lifesaving intervention sooner than current monitoring equipment allows. An optical fiber edema probe could be incorporated into a variety of commonly used or conventional intracranial monitoring devices, including pressure transducers, ventricular drainage catheters, tissue oxygenation probes, or inserted as a standalone device into an area of interest. The scattering and absorption related changes would then be analyzed to provide an edema index on a continuous basis to aid physicians in clinical decision making.

The illustrated embodiment of the invention includes a method for using optical parameters to monitor for a physiological event and/or a state prior to the physiological event comprising the steps of: illuminating neural tissue with diagnostic light of a predetermined frequency at a predetermined location; detecting magnitude of optical scattering by neural tissue of the diagnostic light as a function of time; and determining a signature signal of the optical scattering of the diagnostic light before the physiological event in the neural tissue becomes clinically manifested. In the illustrated embodiment, the physiological events which are featured include cerebral seizures and edema. However, it must be expressly understood that the scope of the invention need not be so limited and may include other types of neural events, vascular events and strokes.

In one embodiment the physiological event is a seizure and the method further comprises the step of mediating the neural activity of the neural tissue before onset of a seizure upon a determination of the signature temporal pattern, such as preventing or reducing symptoms of the seizure.

In one embodiment the step of determining a signature signal of the optical scattering of the diagnostic light comprises determining a threshold value of the optical scattering, including determining a threshold value during one or more time windows of decreasing optical scattering.

In another embodiment the step of determining a signature signal of the optical scattering of the diagnostic light comprises determining a threshold value of the time derivative of the optical scattering, including determining a threshold value of the time derivative of the optical scattering during one or more time windows of decreasing optical scattering.

In yet another embodiment the method further comprises the step of illuminating neural tissue with diagnostic light of a predetermined frequency over a spatial region using spatially modulated light, detecting magnitude of optical scattering of the spatially distributed light diagnostic light as a function of time; and determining a signature signal of the optical scattering of the diagnostic light at each location in the spatial region to image and map regions of brain tissue undergoing seizure.

The illustrated embodiment may also be characterized as a method for using optical parameters in detection of seizure and pre-seizure states comprising the steps of: illuminating neural tissue with diagnostic light of a predetermined frequency at a predetermined position; detecting magnitude of optical scattering by neural tissue of the diagnostic light as a function of time; and detecting changes in neural architecture that result from water and ion migration preceding and during specific brain electrical activity.

The illustrated embodiment may still further be characterized as a method for using optical parameters in detection of seizure and pre-seizure states comprising the steps of: illuminating neural tissue with diagnostic light of a predetermined frequency at a predetermined position; detecting magnitude of optical scattering by neural tissue of the diagnostic light as a function of time; and detecting changes in cell volume and the extracellular space which decreases optical scattering through affected neural tissue.

In an embodiment the diagnostic light is supplied through a source fiber and where the signature signal is detected through a detector fiber comprising arranging and configuring the source and detector fibers to delineate between signal changes due to altered perfusion from signal changes due to changes in cellular architecture.

In another embodiment the diagnostic light is supplied through a source fiber and the signature signal is detected through a detector fiber, and where the source and detector fibers are provided with a plurality of optical apertures longitudinally distributed along the length of the fiber, comprising obtaining measurements of optical scattering of the diagnostic light vertically within deep brain structures.

In one embodiment the diagnostic light is supplied through a source fiber and the signature signal is detected through a detector fiber, the method comprising the step of implanting the source and detector fibers into seizure foci of patients with epilepsy for use as an early warning device.

In the one embodiment the method further comprises the step of illuminating the neural tissue with either broadband or specific wavelengths of radiation in the visible, near-infrared, and/or infrared region, and where determining a signature signal comprises measuring changes in signal intensity associated with a seizure or a pre-seizure activity with a detector.

In yet another embodiment the method comprises the step of using thin implantable optical fibers for implantation into a selected region of a brain to be monitored for delivery of the diagnostic light and return of the signature signal, where a configuration of the location of the diagnostic light and the detected signature signal by the optic fibers and a wavelength of the diagnostic light is selected to be sensitive to selected type of optical change in the brain.

In still another embodiment the method comprises the step of using a single multimode bifurcated fiber to convey both the diagnostic light from a source and return the signature signal to a detector to measure diffuse reflectance, where a close source-detector separation provided by the single multimode bifurcated fiber correlates with changes in the optical scattering coefficient of the monitored neural tissue.

In one embodiment the method further comprises the step of providing an early warning of pathologic brain swelling before measurable late sequelae of increased intracranial pressure or hemodynamic changes.

In other embodiments the method further comprises the step of using an optical fiber edema probe incorporated into an intracranial monitoring device or inserted as a standalone probe into an area of interest in the neural tissue, and analyzing optical scattering and absorption related changes to provide an edema index on a continuous basis.

The illustrated embodiment is also defined as a method for using optical parameters to monitor for a physiological event and/or a state prior to the physiological event comprising the steps of: illuminating neural tissue with diagnostic light of a predetermined frequency at a predetermined position; detecting magnitude of optical scattering and/or optical absorption by neural tissue of the diagnostic light as a function of time; and determining a signature signal of the optical scattering and/or optical absorption of the diagnostic light before the physiological event in the neural tissue becomes clinically manifested.

The illustrated embodiment also includes an apparatus for using optical parameters to monitor for a physiological event and/or a state prior to the physiological event comprising: a source of diagnostic light of a predetermined frequency for illuminating neural tissue at a predetermined location; a detector of optical scattering and/or optical absorption by neural tissue of the diagnostic light as a function of time; and a signal processor for determining a signature signal of the optical scattering and/or optical absorption of the diagnostic light before the physiological event in the neural tissue becomes clinically manifested.

The illustrated embodiment further comprises a thin optical source fiber coupled to the source for delivering the diagnostic light to the predetermined location and a thin optical detector fiber coupled to the detector for returning the signature signal to the detector, the source and detector fibers arranged and configured for implantation into the neural tissue.

In one embodiment the source and detector fibers comprise a single multimode bifurcated fiber.

In another embodiment the apparatus further comprises a noise filtration fiber optically coupled to the source fiber for receiving a portion of the diagnostic signal as delivered to the neural tissue and coupled the received portion to the signal processer for noise filtration.

In still a further embodiment the source and detector fiber are integrated into a medical probe or catheter used for a separate treatment mediation.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a diffuse reflectance reference or non-seizure frame of mouse cortex taken in a laboratory demonstration of the operability of the invention. Two cortical EEG recording electrodes are visible in left of the frame.

FIG. 2 is a graph of the optical scattering coefficient (y-axis) in the lowest data curve and two time-referenced EEG traces in the two upper data curves of the experiment on the mouse cortex of FIG. 1, which demonstrates a measurable decline in optical scattering coefficient following convulsant administration (PTZ) but prior to electrographic seizure onset (SZ) as evidenced by the EEG traces. Seizure termination followed pentobarbital administration (PB) and was accompanied by an increase in the optical scattering coefficient.

FIG. 3 is a graph of absorption-derived total hemoglobin concentration (THC) (y-axis) vs. time (x-axis), which shows an imaging artifact at the time of PTZ injection followed by an abrupt rise in THC after seizure onset.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The apparatus of the illustrated embodiment measures diffuse reflectance, which at close source-detector fiber separations has been shown to correlate strongly with changes in the scattering coefficient of the brain tissue 12. This is the first study to describe the individual contribution of light scattering to the optical signal change before and during seizure activity. Our findings provide proof of principle for optical detection of a pre-seizure state on a clinically relevant timescale.

The illustrated embodiment of the invention in a laboratory demonstration as diagrammatically depicted in FIG. 4 utilizes a source 10, which illuminates the brain tissue 12 with either broadband or specific wavelengths of radiation in the visible, near-infrared, and/or infrared region, through a delivery optic fiber 14 and a detector 16 through an optic fiber 18 to measure changes in signal intensity associated with seizure or pre-seizure activity. The source 10 and detector 16 are connected to thin implantable optical fibers 14 and 18 which allow for any region of brain 12 to be measured. Fibers 14, 18 may employ any diameter optical fibers which allow acceptable optical performance with minimal invasive impact, and in the illustrated embodiment are preferably in the range of 100 to 400 microns in diameter.

The relative configuration of the fibers 14 and 18 coupled to source 10 and detector 16 and the various wavelengths of light used make the apparatus particularly sensitive to certain types of optical changes as is empirically determined, i.e. fiber separations and wavelengths are chosen to preferably select different types of seizure activity, brain location or edema. Alternatively, a single, multimode bifurcated fiber (not shown) may be used to convey the diagnostic light from source 10 and return the scattered light signal to the detector 16.

Our experimental findings indicate that the magnitude of optical scattering of light through brain tissue 12 decreases several minutes before clinical or electroencephalographic (EEG) seizure onset. Thus the process of detecting seizure associated scattering changes provides an important tool for detection of the pre-seizure state which provides a therapeutic window for seizure prevention. The pre-onset signature signal thus is an empirically determined decrease of the magnitude of optical scattering signal during one or more pre-seizure time windows, or an empirically determined magnitude of the rate of decrease or time derivative of the magnitude of the optical scattering signal during one or more pre-seizure time windows.

The illustrated embodiment allows for detection of changes in the optical scattering coefficient of brain tissue 12 through the use of optical fibers 14, 18. In the illustrated embodiment, one fiber 14 illuminates the tissue from a source 10, the other fiber 18 collects light that is scattered by the local tissue 12 and returns it to a detector 16. The fiber cores are separated by approximately 200 microns, a distance which makes the light path highly susceptible to scattering changes that occur during physiologic events, such as cerebral edema or glial swelling during the pre-seizure state. The distance of separation of the location where the source light is injected into the brain tissue and the location where the scattered light is detected may be varied according to empirical adjustments according to the physiologic event which is being monitored.

Our research demonstrates scattering changes are best detected using near-infrared (NIR) light in the 800-900 nm range. Again the wavelength selection and the bandwidth of the diagnostic signal can be varied according to empirical adjustment according to the physiological event and/or location which is being monitored. Accordingly, the device of FIG. 4 in the illustrated embodiment utilizes a broadband NIR source 10 and a photodiode array spectrometer 16 to isolate the wavelengths of interest. In clinical or commercial embodiments the wavelength may be narrowly selected and the components used for the source 10 and detector 16 selected according to the application. For example, in a portable monitoring device, it is to be expected that source 10 may be an LED and detector 16 a filtered photodetector or the equivalent.

Another embodiment of FIG. 4 utilizes an 850 nm diode laser as the source 10 and an avalanche photo diode array (PDA) optical power meter as the detector 16. This allows for a highly stable and more efficient light source 10, more sensitive detection, and faster sampling rates. In the future, it should be possible to utilize even more efficient sources, such as narrow band light-emitting diodes in the NIR range, making self-contained implantable devices feasible.

The probe 22 of the apparatus comprises a plurality optical fibers 14, 18 ensheathed in a bio-compatible covering, such as silastic, with a polished transparent window at the aperture of the fibers 14, 18. The probe 22 in FIG. 4 is held securely within the brain by rigid fixation to the skull with a bolt or friction fit cap screwed to the bone. Again in clinical or commercial embodiments probe 22 will be integrated into other medical devices which are normally employed in the specific application. The probe 22 can be implanted into the brain in much the same fashion as ICP monitors, ventricular catheters, or depth electrodes are currently. In various embodiments of probe 22 it may incorporate multiple source/detector fiber pairs 14, 18 with apertures along the longitudinal fiber length to allow for measurement of an “optical cross-section” at several points along the vertical or longitudinal insertion track of the probe.

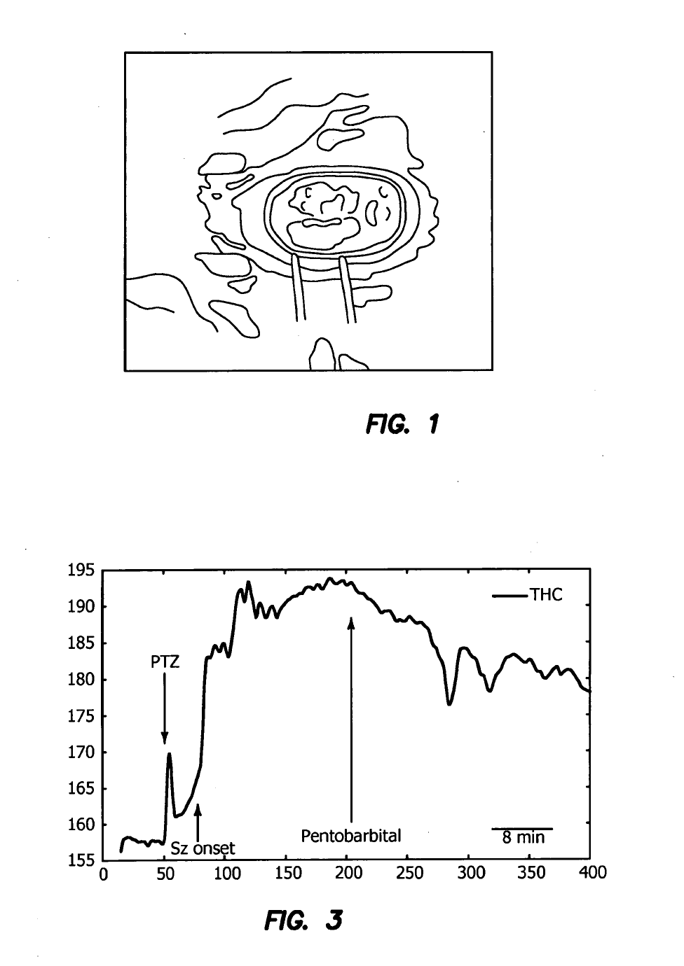

While changes in diffuse cortical reflectance are well described in both animal and human seizures, the individual contributions of absorption and scattering have not hitherto been explored. In another embodiment the device may use spatial modulation of near-infrared light to separately and quantitatively map absorption and scattering changes during seizures or other physiological events. Cranial windows were created in mice, and images were obtained every 5.3-9.7 seconds at 750 and 850 nm using a spatial frequency of 0.34 mm⁻¹ The data image is shown in FIG. 1. Post-processing software (Matlab) is used to generate maps and time plots of chromophore concentrations and scattering coefficients from the absorption and scattering data.

Generalized seizures were induced in mice using the GABA antagonist pentylenetetrazol (PTZ) (100 mg/kg, IP). Seizures were reversed with pentobarbital (30 mg/kg, IP). Continuous electroencephalography (EEG) recordings were obtained via two conventional cortical tungsten microelectrodes 20 connected to a differential amplifier 24 and processed through waveform analysis software (AcqKnowledge, Biopac Systems Inc.) in signal processor 26. Ictal onset was determined by an observer blinded to the optical data.

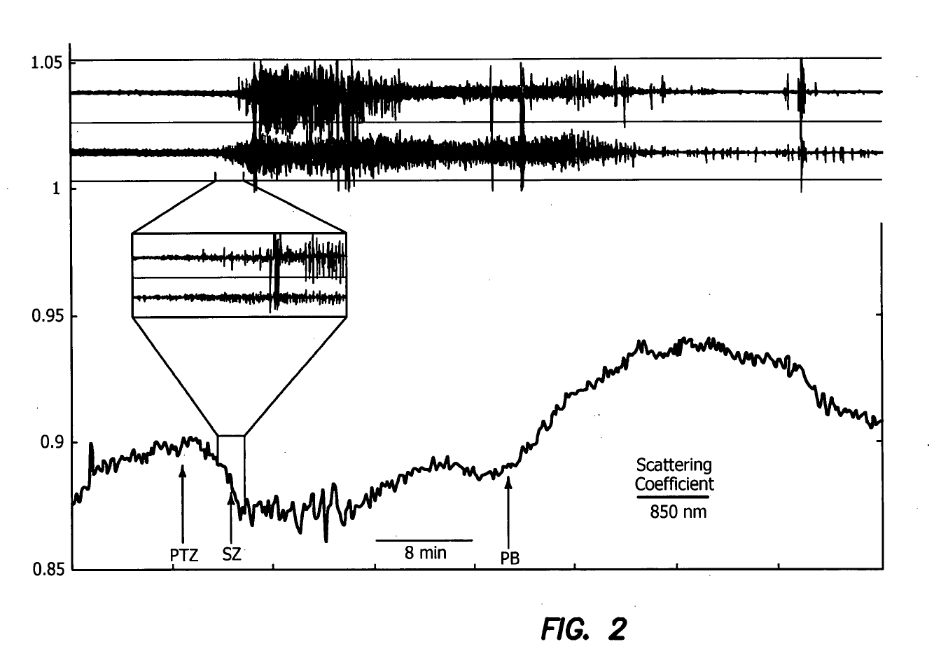

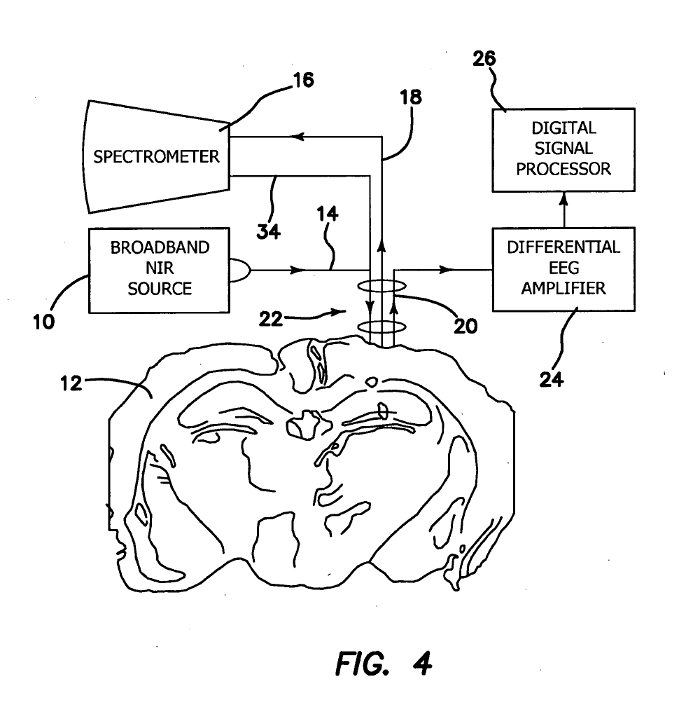

PTZ injection reliably produced clinical and electrographic generalized seizures 3-5 minutes post-injection. In each case, a marked decrease in the scattering coefficient preceded electrographic seizure onset by up to 60 seconds, with a further precipitous decrease following seizure onset as shown in curve 28 in the graph of FIG. 2. This scattering change returned to baseline following seizure termination in region 30. Seizure onset was also coupled to a progressive rise in local hemoglobin concentration, consistent with seizure-induced hyperemia as shown by curve 32 in the graph of FIG. 3.

We have also designed a noise reduction feature that can filter extraneous signals caused by movement of the optical fibers 14, 18, changes in source power, etc. As shown in laboratory setup of FIG. 4 the feature includes a reference fiber 34 added to the source/detector fiber probe 22 including fibers 14, 18, which fiber 34 diverts a portion of the light delivered from the diagnostic signal light delivered by fiber 14 coupled to source 10 to a reflective material (not shown) contained within the probe 22. Light from the reflective material is diverted to detector 16 through the separate reference fiber 34. Reference fiber 34 also receives reflected light signals from the brain tissue 12 like detector fiber 18. Signal processing algorithms stored in a digital signal processor included in detector 16 are used to compare the reflected brain signal to the reference signal reflected from source 10 for noise filtering.

Further signal processing beyond simple measurement of light intensity can also be applied to the reflected signal from brain tissue 22 through fiber 18. For example, a fast computational algorithm based on scattering slope detection or time rate measurements is used for rapid visualization of the optical scattering signal change to create a “trigger” to generate an alert of a possible impending seizure onset. Similar neural trends can be monitored with different temporal sampling windows to provide early warning of increasing cerebral edema before other physiologic sequelae are detectable.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method for using optical parameters to monitor for a physiological event and/or a state prior to the physiological event comprising:
illuminating neural tissue with diagnostic light of a predetermined frequency at a predetermined location;
detecting magnitude of optical scattering by neural tissue of the diagnostic light as a function of time; and
determining a signature signal of the optical scattering of the diagnostic light before the physiological event in the neural tissue becomes clinically manifested.

2. The method of claim 1 where the physiological event is a seizure and further comprising mediating the neural activity of the neural tissue before onset of a seizure upon a determination of the signature temporal pattern.

3. The method of claim 2 where mediating the neural activity of the neural tissue comprises preventing the seizure.

4. The method of claim 2 where mediating the neural activity of the neural tissue comprises reducing the seizure.

5. The method of claim 1 where determining a signature signal of the optical scattering of the diagnostic light comprises determining a threshold value of the optical scattering.

6. The method of claim 5 where determining a threshold value of the optical scattering comprises determining a threshold value during one or more time windows of decreasing optical scattering.

7. The method of claim 1 where determining a signature signal of the optical scattering of the diagnostic light comprises determining a threshold value of the time derivative of the optical scattering.

8. The method of claim 7 where determining a threshold value of the of the time derivative of the optical scattering comprises determining a threshold value of the time derivative of the optical scattering during one or more time windows of decreasing optical scattering.

9. The method of claim 1 further comprising illuminating neural tissue with diagnostic light of a predetermined frequency over a spatial region using spatially modulated light, detecting magnitude of optical scattering of the spatially distributed light diagnostic light as a function of time; and determining a signature signal of the optical scattering of the diagnostic light at each location in the spatial region to image and map regions of brain tissue undergoing seizure.

10. A method for using optical parameters in detection of seizure and pre-seizure states comprising:
illuminating neural tissue with diagnostic light of a predetermined frequency at a predetermined position;
detecting magnitude of optical scattering by neural tissue of the diagnostic light as a function of time; and
detecting changes in neural architecture that result from water and ion migration preceding and during specific brain electrical activity.

11. A method for using optical parameters in detection of seizure and pre-seizure states comprising:
illuminating neural tissue with diagnostic light of a predetermined frequency at a predetermined position;
detecting magnitude of optical scattering by neural tissue of the diagnostic light as a function of time; and
detecting changes in cell volume and the extracellular space which decreases optical scattering through affected neural tissue.

12. The method of claim 1 where the diagnostic light is supplied through a source fiber and where the signature signal is detected through a detector fiber comprising arranging and configuring the source and detector fibers to delineate between signal changes due to altered perfusion from signal changes due to changes in cellular architecture.

13. The method of claim 1 where the diagnostic light is supplied through a source fiber and the signature signal is detected through a detector fiber, and where the source and detector fibers are provided with a plurality of optical apertures longitudinally distributed along the length of the fiber, comprising obtaining measurements of optical scattering of the diagnostic light vertically within deep brain structures.

14. The method of claim 1 where the diagnostic light is supplied through a source fiber and the signature signal is detected through a detector fiber, comprising implanting the source and detector fibers into seizure foci of patients with epilepsy for use as an early warning device.

15. The method of claim 1 further comprising illuminating the neural tissue with either broadband or specific wavelengths of radiation in the visible, near-infrared, and/or infrared region, and where determining a signature signal comprises measuring changes in signal intensity associated with a seizure or a pre-seizure activity with a detector.

16. The method of claim 1 comprising using thin implantable optical fibers for implantation into a selected region of a brain to be monitored for delivery of the diagnostic light and return of the signature signal, where a configuration of the location of the diagnostic light and the detected signature signal by the optic fibers and a wavelength of the diagnostic light is selected to be sensitive to selected type of optical change in the brain.

17. The method of claim 1 comprising using a single multimode bifurcated fiber to convey both the diagnostic light from a source and return the signature signal to a detector to measure diffuse reflectance, where a close source-detector separation provided by the single multimode bifurcated fiber correlates with changes in the optical scattering coefficient of the monitored neural tissue.

18. The method of claim 1 further comprising providing an early warning of pathologic brain swelling before measurable late sequelae of increased intracranial pressure or hemodynamic changes.

19. The method of claim 1 further comprising using an optical fiber edema probe incorporated into an intracranial monitoring device or inserted as a standalone probe into an area of interest in the neural tissue, and analyzing optical scattering and absorption related changes to provide an edema index on a continuous basis.

20. A method for using optical parameters to monitor for a physiological event and/or a state prior to the physiological event comprising:
illuminating neural tissue with diagnostic light of a predetermined frequency at a predetermined position;
detecting magnitude of optical scattering and/or optical absorption by neural tissue of the diagnostic light as a function of time; and
determining a signature signal of the optical scattering and/or optical absorption of the diagnostic light before the physiological event in the neural tissue becomes clinically manifested.

21. An apparatus for using optical parameters to monitor for a physiological event and/or a state prior to the physiological event comprising:
a source of diagnostic light of a predetermined frequency for illuminating neural tissue at a predetermined location;
a detector of optical scattering and/or optical absorption by neural tissue of the diagnostic light as a function of time; and
a signal processor for determining a signature signal of the optical scattering and/or optical absorption of the diagnostic light before the physiological event in the neural tissue becomes clinically manifested.

22. The apparatus of claim 21 further comprising a thin optical source fiber coupled to the source for delivering the diagnostic light to the predetermined location and a thin optical detector fiber coupled to the detector for returning the signature signal to the detector, the source and detector fibers arranged and configured for implantation into the neural tissue.

23. The apparatus of claim 22 where the source and detector fibers comprise a single multimode bifurcated fiber.

24. The apparatus of claim 22 further comprising a noise filtration fiber optically coupled to the source fiber for receiving a portion of the diagnostic signal as delivered to the neural tissue and coupled the received portion to the signal processer for noise filtration.

25. The apparatus of claim 22 where the source and detector fiber are integrated into a medical probe or catheter used for a separate treatment mediation.
